(19) United States
(12) Patent Application Publication
(10) Pub. No.: US 2017/0095625 A1
(43) Pub. Date: Apr. 6, 2017

(54) DRY POWDER INHALER WITH DOSE DEPLETION EVALUATION

(71) Applicant: MERCK SHARP & DOHME CORP., RAHWAY, NJ (US)

(72) Inventors: PETER A. BASILE, BLOOMSBURY, NJ (US); MICHAEL GALLUPPI, MIDDLESEX, NJ (US); MIKHAIL GOTLIBOYM, SCOTCH PLAINS, NJ (US); CHRISTOPHER GRANELLI, CHATHAM, NJ (US); SCOTT BROWN, PRINCETON, NJ (US)

(73) Assignee: MERCK SHARP & DOHME CORP., RAHWAY, NJ (US)

(21) Appl. No.: 15/315,988

(22) PCT Filed: May 29, 2015
(86) PCT No.: PCT/US15/33095

§ 371 Date: Dec. 2, 2016

Related U.S. Application Data
(60) Provisional application No. 62/007,653, filed on Jun. 4, 2014.

(51) Int. Cl. A61M 15/00

(52) U.S. Cl. CPC A61M 15/008; A61M 15/0091; A61M 15/0028; A61M 2202/064; A61M 2205/3306; A61M 2205/3331; A61M 2205/3317; A61M 2205/505; A61M 2205/584

(57) ABSTRACT

A dry powder inhaler is provided herein which includes a dose chamber having a staging area configured to accommodate an inhalable dose of active pharmaceutical agent. A nozzle is provided having a discharge aperture with an inhalation channel communicating the staging area with the discharge aperture. The inhalation channel is configured such that sufficient negative pressure applied to the discharge aperture draws a dose from the staging area towards the discharge aperture. An arrangement is provided for evaluating the level of depletion of a dose from the staging area. Advantageously, the subject invention allows for evaluating physical depletion of a staged dose so as to recognize the level of delivery thereof.

### FIELD OF THE INVENTION

The invention relates to dry powder inhalers, drug products and, more particularly, dry powder inhalers with dose depletion evaluation.

### BACKGROUND OF THE INVENTION

Various devices have been used to dispense inhaled metered doses of active pharmaceutical agent (APA). Dry powder inhalers (DPI's) dispense metered doses of powdered medicament by inhalation. DPI designs may be found in U.S. Pat. No. 6,240,918, U.S. Pat. No. 5,829,434, U.S. Pat. No. 5,394,868 and U.S. Pat. No. 5,687,710, which are all incorporated by reference herein.

It is noted that with DPI's, due to the fineness of delivered powder, a user may not be aware if a full dose has been delivered or not. This may lead to partial, and possibly no, dose delivery due to a user prematurely stopping inhalation prior to complete delivery of a dose. A dose may be completed without any tactile sensation.

A DPI device has been developed in the prior art which includes a “Dosing Done” indication light. This device utilizes an inhalation sensor that detects a patient's inspiratory airflow. Upon breach of a threshold value, the device relies on an algorithm-controlled piezoelectric construct to disperse the APA for delivery. Completion of a dose is indicated by the end of the operating cycle of the piezoelectric construct. This device does not physically evaluate how much of a dose is actually administered.

### SUMMARY OF THE INVENTION

A dry powder inhaler is provided herein which includes a dose chamber having a staging area configured to accommodate an inhalable dose of active pharmaceutical agent (APA). A nozzle is provided having a discharge aperture with an inhalation channel communicating the staging area with the discharge aperture. The inhalation channel is configured such that sufficient negative pressure applied to the discharge aperture draws a dose from the staging area towards the discharge aperture. An arrangement is provided for evaluating the level of depletion of a dose from the staging area. Advantageously, the subject invention allows for evaluating physical depletion of a staged dose so as to recognize the level of delivery thereof

These and other features of the invention will be better understood through a study of the following detailed description and accompanying drawings.

### BRIEF DESCRIPTION OF THE DRAWINGS

FIG. 1 is a perspective view of a dry powder inhaler formed in accordance with the subject invention;

FIG. 2 shows generally components of a dry powder inhaler useable with the subject invention;

FIG. 3 is a schematic of a staging area useable with the subject invention;

FIG. 4 shows a rupturable blister package useable with the subject invention;

FIG. 5 shows a peel-open foil package useable with the subject invention;

FIGS. 6A-7B show optosensors useable with the subject invention;

FIG. 8 shows pressure sensors useable with the subject invention;

FIG. 9 shows a capacitance sensor useable with the subject invention;

FIG. 10 is a schematic representing electrically powered components useable with the subject invention;

FIG. 11 shows a display useable with the subject invention;

FIGS. 12 and 13 show a modularly assembled dry powder inhaler formed in accordance with the subject invention; and,

FIG. 14 is a graph representing an optical reflectance signal versus time over a dosing cycle.

### DETAILED DESCRIPTION OF THE INVENTION

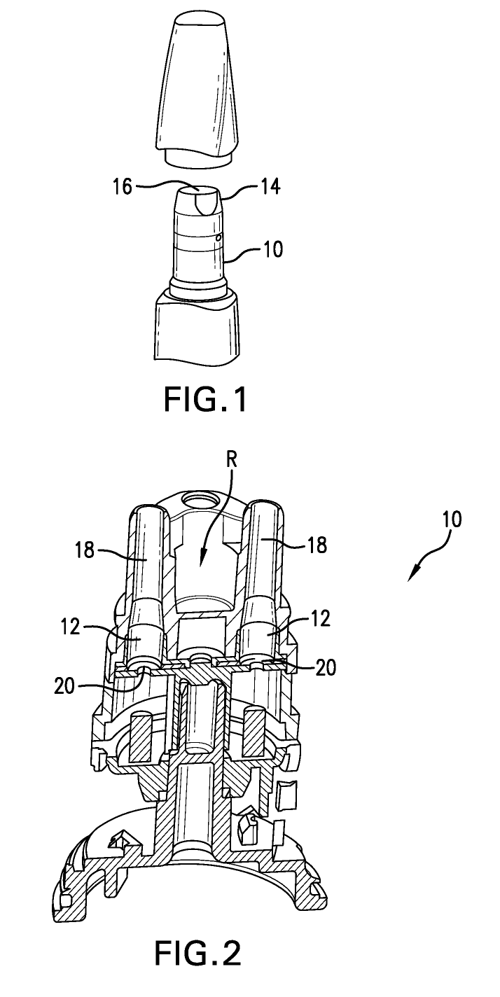

With reference to the FIG. 1, a dry powder inhaler 10 is shown which includes an arrangement for evaluating depletion of the dose so as to recognize the level of delivery thereof. The dry powder inhaler 10 may be of any configuration which depends on inhalation for delivery of an active pharmaceutical agent (APA), including, but not limited to, the designs as shown in U.S. Pat. No. 6,240,918, U.S. Pat. No. 5,828,434, U.S. Pat. No. 5,394,868 and U.S. Pat. No. 5,687,710.

With reference to FIGS. 1 and 2, the dry powder inhaler 10 generally includes at least one dose chamber 12, a nozzle 14 having a discharge aperture 16, and at least one inhalation channel 18. As shown in FIG. 2, a plurality of the dose chambers 12 may be provided each having at least one of the inhalation channels 18. Various components may be provided along the flow path of each inhalation channel 18, such as a swirl nozzle, a deagglomerator, and so forth.

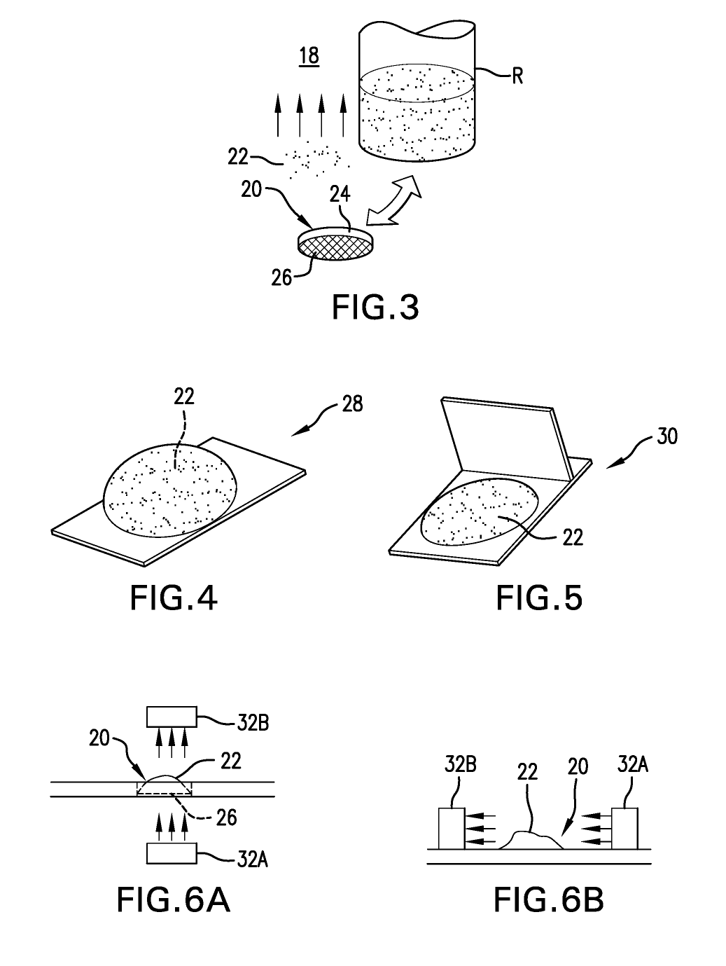

The dose chamber 12 includes at least one staging area 20 formed to accommodate an inhalable dose 22 of active pharmaceutical agent (APA). The dose 22 can be prepared in the staging area 20 in any manner. For example, as set forth in U.S. Pat. No. 6,240,918, and shown schematically in FIG. 3, the staging area 20 may include a recess 24 with a supporting mesh surface 26. The volume of the recess 22 may be used to define the volume of the dose 22. Using any known technique, the staging area 20 may be replenished from a reservoir R containing a plurality of the doses, e.g., in loose powder form, such as by being moved in and out of communication with the reservoir R, e.g., by rotation. Alternatively, pre-separated doses, which may be individually packaged, or otherwise prepared, may be introduced to the staging area 20 as needed for dosing. For example, rupturable blister packages or capsules 26 (FIG. 4) or peel-open foil packages 28 (FIG. 5) may be utilized, each including a unit dose of APA. Any known configuration for introducing rupturable blister packages or capsules and peel-open foil packages may be utilized. As will be appreciated by those skilled in the art, the staging area 20 is a location where the dose 22 is initially located in anticipation of delivery. As shown in FIG. 3, the inhalation channel 18 communicates the staging area 20 with the discharge aperture 16 so that sufficient negative pressure applied to the discharge aperture 16 draws the dose 22 from the staging area 20 towards the discharge aperture 16. This negative pressure is generated by a user inhaling with the nozzle 14 being in the user's mouth and is utilized to deliver the dose 22 to the user.

It is to be understood that reference to a “dose” herein includes a single complete dose, as well, a fraction of a complete dose (e.g., where a plurality of the staging areas 20 are utilized, each providing a fraction of an intended total dose to a patient). Fractional portions of a dose may be combined in one or more of the inhalation channels 18 and/or in the nozzle 14 for delivery.

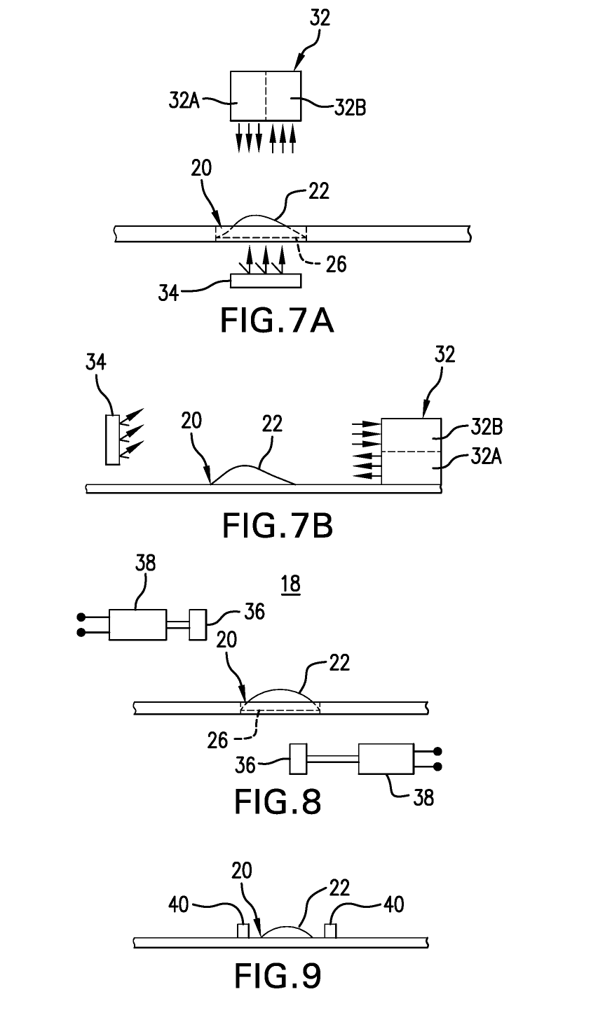

An arrangement is provided with the dry powder inhaler 10 to evaluate the level of depletion of the dose 22 from the staging area 20. This allows for real-time monitoring of depletion of the dose 22 during a dosing cycle to determine the actual level of delivery thereof. Various arrangements for physically evaluating the level of depletion may be utilized. With reference to FIGS. 6A-7B, at least one optosensor 32 may be utilized to observe the level of depletion of the dose 22 from the staging area 20. For example, the optosensor 32 may be a photoelectric sensor which may be of the reflective-type. As will be appreciated by those skilled in the art, the optosensor 32 may be of various configurations. With reference to FIGS. 6A and 6B, the optosensor 32 may include an electromagnetic energy emitter 32A and a corresponding receiver 32B located on opposing sides of the staging area 20. The dose 22 provides varying levels of obstruction of passage of electromagnetic energy between the emitter 32A and the receiver 32B as the dose 22 is administered. As such, the level of depletion of the dose 22 may be determined as a function of the amount of electromagnetic energy detected by the receiver 32B from the emitter 32A. As shown in FIG. 6A, the optosensor 32 may observe the staging area 20 from above or below, particularly where the staging area 20 is transmissive to the electromagnectic energy of the optosensor 32 (e.g., where the mesh surface 26 is utilized). In addition, or alternatively, as shown in FIG. 6B, the optosensor 32 may be located to observe the dose 22 from a side perspective, e.g. in a plane parallel to the staging area 20.

As shown in FIGS. 7A and 7B, the optosensor 32 may have the emitter 32A and 32B located together, e.g., in the same housing. Here, the optosensor 32 may rely on reflectance of the electromagnetic energy off the dose 22 to provide an indication of the level of depletion thereof. The level of reflected electromagnetic energy detected by the receiver 32B may be used to determine the level of depletion of the dose 22. The optosensor 32 may be located above the staging area 20 (FIG. 7A) or to the side of the staging area 20 (FIG. 7B). In addition, the optosensor 32 may be located below the staging area 20 if the staging area 20 is transmissive to the electromagnectic energy of the optosensor 32 (e.g., where the mesh surface 26 is utilized). The optosensor 32 may optionally include a reflector 34 positioned to reflect the electromagnetic energy from the emitter 32A to the receiver 32B. If utilized, the reflector 34 is positioned on the opposite side of the staging area 20 away from the optosensor 32. Reflectance from the reflector 34 is primarily relied upon, if utilized, rather than reflectance from the dose 22.

In addition, or alternatively, as shown in FIG. 8, at least one pressure sensor 36 may be provided for measuring pressure in proximity to the staging area 20. Any known pressure sensor may be utilized, including a mechanical pressure gauge, piezoelectric sensor and so forth. The pressure sensor 36 may include a transducer 38 to convert pressure readings into digital format. The pressure sensor 36 may be located on the opposing side of the staging area 20 away from the inhalation channel 18 so that the staging area 20 is located in between the pressure sensor 36 and the inhalation channel 18. This allows for the pressure sensor 36 to detect pressure past the staging area 20 during dose delivery to provide an indication of actual pressure sensed at the staging area 20. This may be particularly effective where the mesh surface 26 is utilized in the staging area 20. Alternatively, the pressure sensor 36 may be located adjacent to the staging area 20 on the same side of the staging area 20 as the inhalation channel 18. Further, two of the pressure sensors 36 may be provided on opposing sides of the staging area 20 to sense pressure drops thereacross. Sensed pressure levels on one or both sides of the staging area 20, based on calculations and/or empirical data, may be used as indicators of physical depletion of a dose from the staging area 20.

Further, in addition, or alternatively, as shown in FIG. 9, at least one capacitance sensor 40 may be located adjacent to the staging area 20 configured to detect changes in levels of capacitance across the staging area 20. Such changes in the level of capacitance may be correlated with calculated or empirical data to indicate levels in change in volume of the dose 22.

In all, physical depletion of a dose from the staging area 20 may be detected with the subject invention. This is in contrast to the prior art which relies on detected levels of inhalation to assume that proper dose delivery is achieved. Actual levels of depletion are not evaluated. As such, improper assumptions or readings with the prior art may provide a false reading that a dose has been completely administered when in fact it has not. With the subject invention, physical depletion of the dose 22 is evaluated to gauge full and complete actual dose delivery. Any combination of one or more of the optosensor 32, pressure sensor 36 and the capacitance sensor 40 may be utilized and shall be referred to as the “arrangements” herein.

The pressure sensor 36 may be provided in conjunction with the optosensor 32 and/or the capacitance sensor 40 to provide detection of inhalation in addition to monitoring of actual physical depletion of the dose 22. In particular, the optosensor 32 and/or the capacitance sensor 40 may be provided to monitor for dose depletion along with the pressure sensor 36 monitoring inhalation. The pressure sensor 36 may be configured to detect a certain pressure level as representative of sufficient inhalation being applied for dosing. This pressure detection provides physical detection of inhalation and aides in avoiding false dose depletion readings. As an example, the dry powder inhaler 10 may be inverted or otherwise positioned to dislodge the dose 22 from the staging area 20 after being readied. The optosensor 32 and the capacitance sensor 40 would detect the staging area 20 as being fully depleted in this event. The pressure sensor 36 allows for the additional detection of inhalation as an additional check to verify proper dose administration. Thus, dose depletion is detected with both detection of physical depletion of the dose 22 and that sufficient inhalation had been applied to the staging area 20.

The arrangements may be utilized with various modes of preparing the dose 22. With the dose 22 being in the rupturable blister package or capsule 26 or the peel-open foil package 28, visual access of the dose 22 may be at least partially obscured by the related packaging material. Dose depletion monitoring may be still achieved by various techniques, such as, the related package may be formed of electromagnetic energy transmissive material to permit dose depletion monitoring by the optosensor 32. In addition, the pressure sensor 36 and/or the capacitance sensor 40 may be utilized. As shown in the Figures, the arrangements are particularly well-suited to evaluate depletion of a dose of loose (unpackaged) APA.

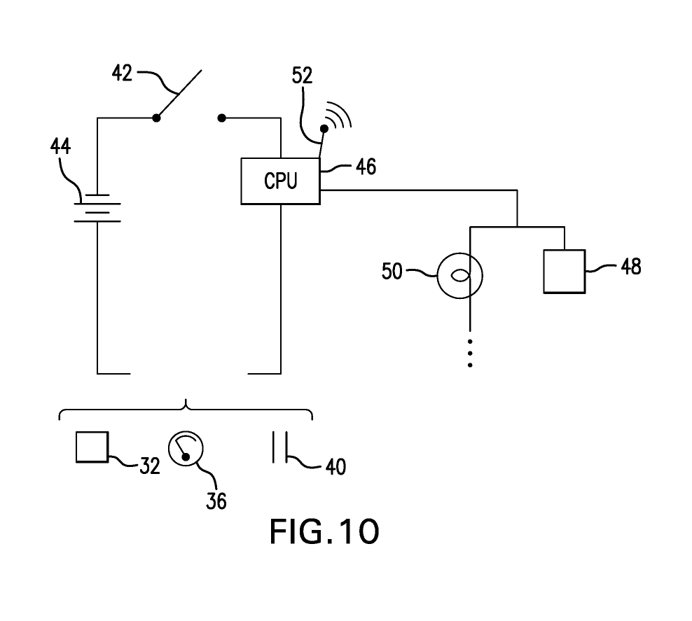

With reference to FIG. 10, once the dose 22 has been staged in the staging area 20 and is ready for administration, a switch 42 may be triggered to place the arrangements into an active state. The arrangements may be maintained in a quiescent state between dosings to conserve power. The switch 42 may be triggered by portion of the staging process in preparing the dose 22 (e.g., removal of a cap, movement of the staging area 20) or by inhalation of a user. The switch 42 may be manual which would require a user to activate. It is possible to continually power the arrangements without requiring the switch 42.

Power source 44 may be provided for electrically powering the arrangements and other components requiring electrical power. The dry powder inhaler 10 may be configured to not require any electrical power for operation thereof in staging a dose and to administer the dose. The power source 44 may be a DC based source, such as a replaceable or chargeable battery.

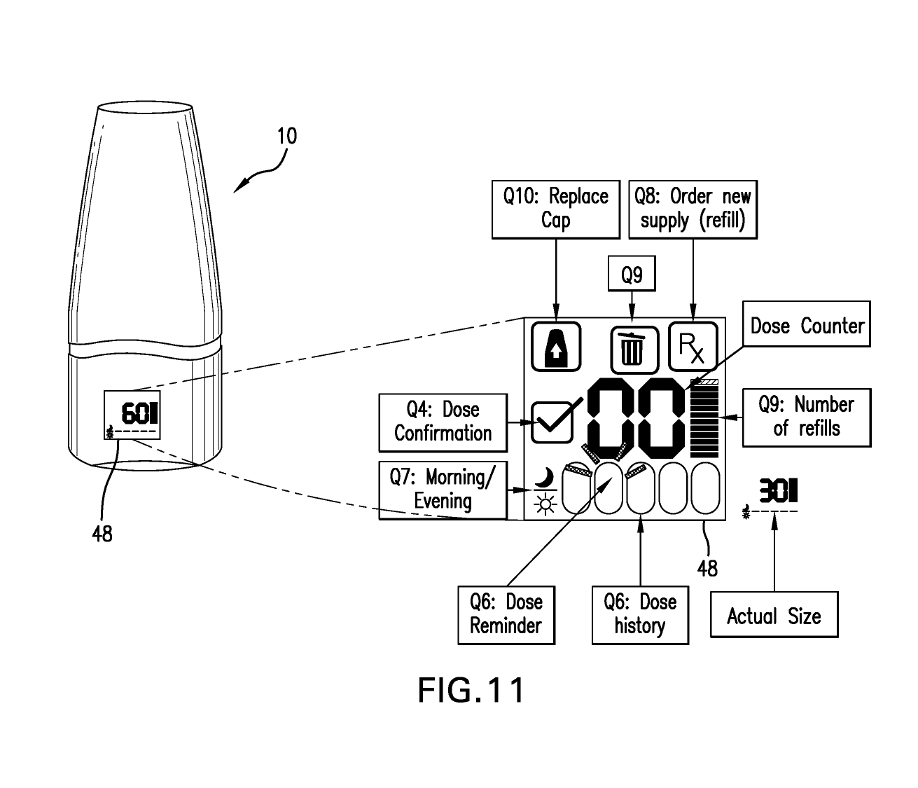

A computer processing unit (CPU) 46 may be electrically coupled with the arrangements to process readings thereof. Power for the CPU 46 may be provided by the power source 44. The CPU 46 may be configured to control display of different states of dosing. The CPU 46 may be linked to a display 48 (FIG. 11) to show different states of the dose 22 from a ready state (staged, ready for delivery) to a completed state based on readings from the arrangements. The display 48 may graphically represent the different states in any manner, such as by textual indications, bar graph, pie graph, percentage representation, and so forth. If the user stops a dosing cycle prior to the arrangements detecting complete depletion of the dose 22, the display 48 may indicate that only a partial dose was administered, thus prompting the user to further inhale. This can continue until there is an indication that the dose 22 has been depleted. In addition to the display 48, or in lieu thereof, one or more indicator lights 50 may be utilized to represent the different states of dosing. Different color lights may be utilized to represent the different states of dosing: green light may be used to represent the ready state; yellow light may be used to represent an incomplete dose; and red light may represent dose completed state. An indication of complete dosing may signal the switch 42 to deactivate the arrangements.

The CPU 46 may store readings from the arrangements. The readings may be retrievable from the CPU 46 through hard-wire linking therewith (jack or port connection) or through a wireless connection, such as by wireless transmitter 52, to evaluate compliance with a dosing regimen. In addition, the CPU 46 may be configured to perform other functionality such as dose counting. The CPU 46 may include a counter to keep count of each completed dose. With a specified number of total available doses, a low supply warning may be provided to the user, such as by a graphic on the display 48 and/or by an indicator light 50. Further, the CPU 46 may be configured to keep track of a user's dosing regimen and provide dosing reminders. A clock may be provided with the CPU 46 to facilitate dose schedule tracking.

A user interface may be provided to allow a user to enter data into the CPU 46. This allows for a user to enter their dosing regimen and other personalized information. The user interface may be application software prepared for a smartphone, or other device, which can communicatively couple with the CPU 46, such as wirelessly (e.g., through a blue tooth connection) or through a hard-wired connection. In addition, or alternatively, the display 48 may be a graphical user interface (GUI) which may be touch-enabled to accept input. Other interfaces may be utilized such as buttons.

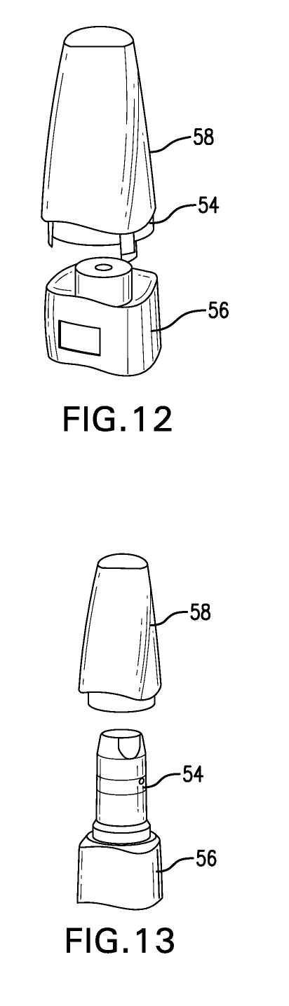

With reference to FIGS. 12 and 13, the dry powder inhaler 10 may be modularly formed by a drug module 54 and an electronics module 56. The arrangements along with the power source 44, and all other components requiring electrical power from the power source (the CPU 46, the display 48, the lights 50, the wireless transmitter 52), may be located in the electronics module 56 so as to be isolated therein. This allows for reusability of the electronic components. The drug module 54 may include elements needed to stage the dose 22, along with delivery components (the nozzle 14, the discharge aperture 16, the inhalation channel 18, the reservoir R). For use, the drug module 54 may be coupled to the electronics module 56 with subsequent doses being staged by the drug module 54 and depletion of the doses being evaluated by the electronics module 56. The power source 44 may be sized to allow a single electronics module 56 to be used with a plurality of drug modules 54. This avoids the need to discard electrical components with exhaustion of APA from a dry powder inhaler.

The drug module 54 and the electronics module 56 may be configured to couple together, preferably releasably, using any known configuration. The coupling may be configured to provide information to the CPU 46 regarding the corresponding drug module 54. For example, different pin patterns may be provided on the drug module 54 to be received by the electronics module 56 with the different pin patterns providing particular details about the drug module 54 (e.g., number of available doses, set dosing regimen). In this manner, different drug modules may be utilized with the electronics module 56 with little to no loss of functionality.

Portions of the arrangements may be located in the drug module 54 (e.g., reflector 34). It is preferred that the arrangements be wholly contained within the electronics module 56. This allows for the electronics module 56 to be a fully stand-alone unit. By way of non-limiting example, the electronics module 56 may contain at least one of the optosensors 32 configured to utilize reflectance of electromagnetic energy off the dose 22 with the dose 22 being located in the drug module 54. At least one of the pressure sensors 36 may be also located in the electronics module 56 to provide for inhalation detection, as discussed above, as an additional check on proper dose delivery.

A cap 58 may be provided which is securable to the dry powder inhaler 10 to cover the discharge aperture 16. The cap 58 may be securable to the drug module 54, if utilized.

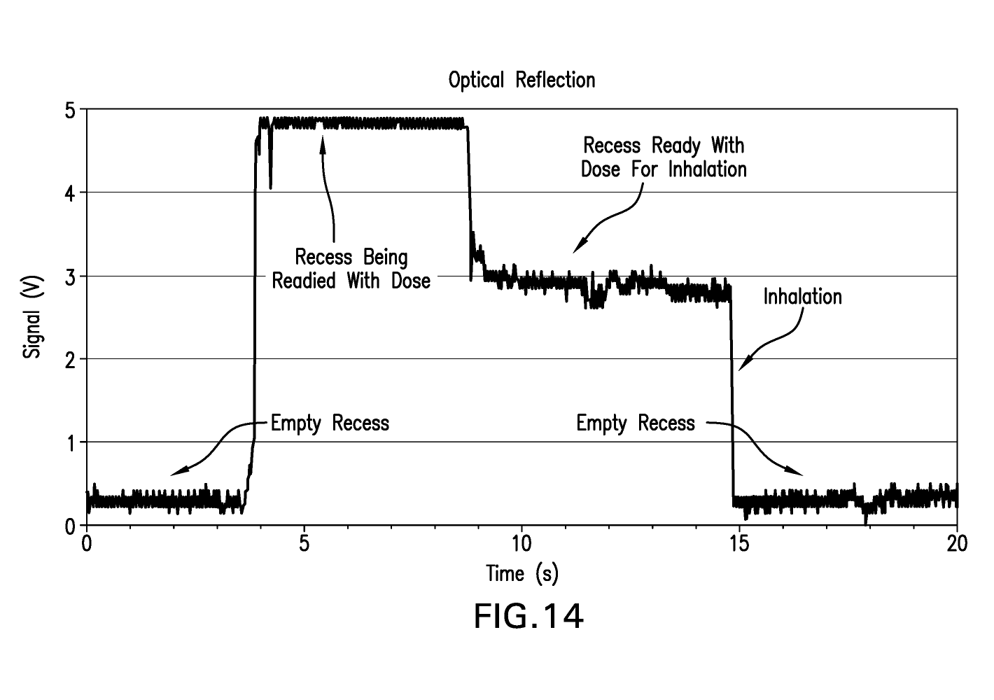

FIG. 14 is a graph showing a possible operating cycle for the arrangements. Here, one of the optosensors 32 is utilized to reflect and detect electromagnetic energy directly off the dose 22. The staging area 20 is configured as shown in FIG. 3 to include the recess 24, the mesh surface 26 and movement in and out of alignment with the reservoir R for dose replenishment. As shown in FIG. 14, the optosensor 32 detects the recess 24 as being empty (minimal reflectance) at the beginning and end of a dosing cycle, i.e., the dose 22 is not present in the recess 24. High reflectance is detected with the recess 24 moved into alignment with the reservoir R, once the recess 24 is out of alignment with the optosensor 32. The solid surface surrounding the staging area 20 causes the high reflectance. With the recess 24 rotated back to a ready state, the dose 22 provides an intermediate level of reflectance indicative of a full dose. With inhalation, the reflectance diminishes to a low state indicative of dose depletion. This cycle may be repeated.

In addition, efficiency of the administration of a dose may be determined. Rate of air flow through the inhalation channel 18 (e.g., by spaced-apart pressure sensors, etc.) may be monitored and compared against stored empirical data to determine the efficiency of the administration of the dose. Better efficiency indicates deeper delivery of the particles of the dose 22 into the lungs of a patient. In addition, or alternatively, one or more optical sensors may be placed along the inhalation channel 18 to monitor velocity of the particles of the dose 22. The measured particle velocity can likewise be compared with empirical data to determine the efficiency of the administration of the dose.

What is claimed is:

1. A dry powder inhaler comprising:
dose chamber including a staging area configured to accommodate an inhalable dose of active pharmaceutical agent;
nozzle having a discharge aperture;
inhalation channel communicating said staging area with said discharge aperture, said inhalation channel configured such that sufficient negative pressure applied to said discharge aperture draws a dose from said staging area towards said discharge aperture; and,
means for evaluating the level of depletion of a dose from said staging area.

2. A dry powder inhaler as in claim 1, wherein said means for evaluating includes at least one optosensor.

3. A dry powder inhaler as in claim 1, wherein said means for evaluating includes at least one pressure sensor in proximity to said staging area.

4. A dry powder inhaler as in claim 3, wherein said staging area is interposed between said pressure sensor and said inhalation channel.

5. A dry powder inhaler as in claim 4, wherein said staging area includes a mesh surface positioned to support a dose.

6. A dry powder inhaler as in claim 1, wherein said means for evaluating includes at least one capacitance sensor.

7. A dry powder inhaler as in claim 1, wherein said staging area is defined by a recess.

8. A dry powder inhaler as in claim 7, wherein said staging area includes a mesh surface adjacent the recess positioned to support a dose.

9. A dry powder inhaler as in claim 1, wherein said dose chamber, said nozzle and said inhalation channel are located in a first module and said means for evaluating is located at least partially in a second module, said first and second modules being coupleable.
